(19) United States
(12) Patent Application Publication
(10) Pub. No.: US 2022/0301662 A1
(43) Pub. Date: Sep. 22, 2022

(54) INFORMATION PROCESSING SYSTEM, INFORMATION PROCESSING DEVICE, LEARNING DEVICE, INFORMATION PROCESSING METHOD, LEARNING METHOD, AND PROGRAM

(71) Applicant: Tokyo Ohka Kogyo Co., Ltd., Kawasaki-shi (JP)

(72) Inventors: Hitoshi YAMANO, Kawasaki-shi (JP); Ryohei EGUCHI, Kawasaki-shi (JP); Makoto SATO, Kawasaki-shi (JP); Hiroaki SHIMIZU, Kawasaki-shi (JP)

(73) Assignee: Tokyo Ohka Kogyo Co., Ltd., Kawasaki-shi (JP)

(21) Appl. No.: 17/638,154

(22) PCT Filed: Aug. 14, 2020
(86) PCT No.: PCT/JP2020/030885

§ 371 Date: Feb. 24, 2022

(30) Foreign Application Priority Data
Sep. 11, 2019 (JP) 2019-165263

(51) Int. Cl. G16C 60/00; G16C 20/70

(52) U.S. Cl. CPC G16C 60/00; G16C 20/70

(57) ABSTRACT

An information processing system includes a storage unit configured to store correspondence information in which material information indicating a material of a composition and a process condition in a process using the composition are associated with performance information of a composition obtained by the process, a performance estimation unit configured to acquire the performance information on the basis of the input material information and processing condition, and the correspondence information, and an output unit configured to output the performance information.

### TECHNICAL FIELD

The present invention relates to an information processing system, an information processing device, a learning device, an information processing method, a learning method, and a program.

Priority is claimed on Japanese Patent Application No. 2019-165263, filed Sep. 11, 2019, the content of which is incorporated herein by reference.

### BACKGROUND ART

With the advance in nanotechnology, it is required to improve the performance of resists such as photoresists and electron beam (EB) resists. For this reason, a developer has tried to develop a new resist by repeating experiments using various materials (for example, refer to Non-Patent Literature 1).

### CITATION LIST

### Patent Literature

### Non Patent Literature

[Non Patent Literature 1]

Latest developments in photoresist materials and process optimization technology (CMC Publishing, edited by: Akira Kawai)

### SUMMARY OF INVENTION

### Technical Problem

However, since there are many types of material candidates and there are various situations in which a resist is used, a burden on developers who are developing new resists is heavy. In addition, such problems are not limited to a resist, and are common issues in the development of a composition where there are many candidate materials and various situations in which they are used.

In view of the circumstances described above, it is an object of the present invention to provide a technology for reducing the labor of developers developing new compositions.

### Solution to Problem

According to one aspect of the present invention, an information processing system includes a storage unit configured to store correspondence information in which material information indicating a material of a composition and a process condition in a process using the composition are associated with performance information of a composition obtained by the process, a performance estimation unit configured to acquire the performance information on the basis of the input material information and processing condition, and the correspondence information, and an output unit configured to output the performance information.

According to another aspect of the present invention, an information processing device includes a performance estimation unit configured to read correspondence information in which material information indicating a material of a resist and a process condition in a predetermined condition using the resist are associated with performance information indicating a performance of the resist obtained by the process from a storage unit, and to acquire the performance information on the basis of the read material information and process condition and the correspondence information, and an output unit configured to output the performance information.

According to still another aspect of the present invention, a learning device includes a learning unit configured to generate first correspondence information by performing machine learning on the basis of material information indicating a material of a resist and a process condition in a predetermined process using the resist, and physical property information indicating physical properties of the resist in a process under the process condition.

According to still another aspect of the present invention, an information processing method includes a performance estimation step of acquiring performance information on the basis of correspondence information in which material information indicating a material of a resist and a process condition in a predetermined process using the resist are associated with the performance information indicating performance of the resist obtained by the process, and an output step of outputting the performance information.

Another aspect of the present invention is a program for causing a computer to function as the information processing system described above.

According to still another aspect of the present invention, a learning method includes a learning step of generating first correspondence information by performing machine learning on the basis of material information indicating a material of a resist and a process condition in a predetermined process using the resist, and physical property information indicating physical properties of the resist in a process with the material information and the process condition.

Still another aspect of the present invention is a program for causing a computer to function as the learning device described above.

### Advantageous Effects of Invention

According to the present invention, the labor of developers developing new compositions can be reduced.

### BRIEF DESCRIPTION OF DRAWINGS

FIG. 1 is a conceptual diagram for describing an embodiment.

FIG. 2 is a diagram which shows an example of a system configuration of an information processing system of the embodiment.

FIG. 3 is a diagram which shows an example of learning data in the embodiment.

FIG. 4 is a diagram which shows an example of regression data in the embodiment.

FIG. 5 is a diagram which shows an example of a functional configuration of a learning device in the embodiment.

FIG. 6 is a flowchart which shows an example of a flow of processing of generating a first model executed by the learning device in the embodiment.

FIG. 7 is a flowchart which shows an example of a flow of processing of generating a second model executed by the learning device in the embodiment.

FIG. 8 is a diagram which shows an example of estimation target information in the embodiment.

FIG. 9 is a diagram which shows an example of a functional configuration of a control unit in the embodiment.

FIG. 10 is a flowchart which shows an example of a flow of processing executed by the information processing device in the embodiment.

FIG. 11 is a diagram which shows a first example of an experimental result showing a relationship between an estimation result of performance information of the information processing system in the embodiment and actually measured performance.

FIG. 12 is a diagram which shows a second example of the experimental result showing the relationship between an estimation result of performance information of the information processing system in the embodiment and actually measured performance.

### DESCRIPTION OF EMBODIMENTS

Hereinafter, embodiments of the present invention will be described with reference to the drawings.

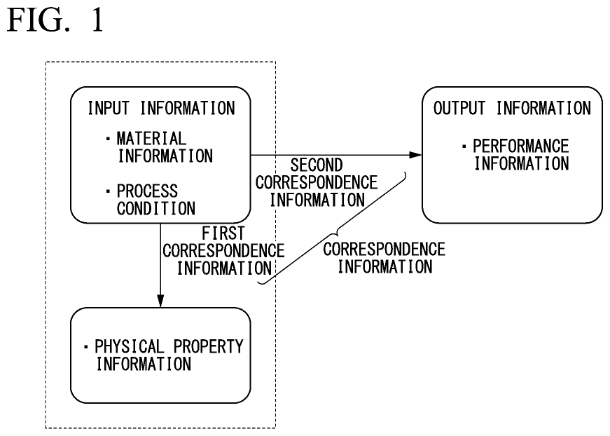

FIG. 1 is a conceptual diagram for describing an embodiment. More specifically, it is a conceptual diagram for describing an information processing system 100 of an embodiment to be described below. The information processing system 100 of the embodiment receives an input of material information indicating a material of a composition and a process condition for a predetermined process for the composition composed of the material indicated by the material information. The composition is, for example, a substance used for patterning an object. More specifically, the composition includes, for example, a resist, a developing solution, a material for forming a phase-separated structure, an etching solution, a cleaning solution, a stripping solution, an optical material, a nanoimprint material, a base material, a hard mask material, a water-repellent material, a composition for forming a separation layer, and an adhesive composition. Among them, a resist is preferable as the composition.

The resist is, for example, a resist for photolithography. The resist may be either a positive type in which an exposed portion of a resist film changes to have a characteristic of dissolving in a developing solution, or a negative type in which the exposed portion of the resist film changes to have a characteristic of not dissolving in a developing solution. In addition, the resist is suitable for lithography using any of an ArF excimer laser, a KrF excimer laser, ghi rays, an F₂ excimer laser, extreme ultraviolet rays (EUV), vacuum ultraviolet rays (VUV), an electron beam (EB), X-rays, and soft X-rays.

The information processing system 100 acquires performance information on the basis of the input material information and process condition and correspondence information stored in advance. The performance information is information indicating the performance of a composition indicated by the material information, which is the performance obtained by a process under the process condition. The performance is, for example, performance (hereinafter referred to as “patterning performance”) when a resist is used for patterning. The patterning performance may be, for example, a lithography performance. The lithography performance is, for example, pattern dimensional variation.

The correspondence information indicates a relationship between material information and a process condition and performance information. The correspondence information includes first correspondence information and second correspondence information.

The first correspondence information is information in which material information and a process condition are associated with physical property information indicating physical properties of a resist in a process with the material information and the process condition. The first correspondence information is information acquired in advance by the information processing system 100 in a predetermined method such as basic four arithmetic operations or machine learning. The first correspondence information is, for example, a learned model that is a learning result of machine learning according to a plurality of learning data including material information and process conditions as learning data on an input side and physical property information as teacher data.

The learned model is a machine learning model when an end condition is satisfied in learning. The end condition may be any condition as long as it is a condition related to an end of learning. The end condition may be, for example, a condition that learning using a predetermined number of data sets has been executed, and the end condition may also be, for example, a condition that the amount of change in parameters due to learning is less than a predetermined magnitude.

The machine learning model refers to a machine learning model in machine learning including deep learning. The machine learning model may be, for example, a neural network of an encoder/decoder model, may be a convolutional neural network, and may also be a gradient boosting determination tree, or reinforcement learning. Learning refers to appropriately adjusting the parameters of the machine learning model. The parameters of the machine learning model are adjusted by an algorithm in an error back propagation method, for example, when the machine learning model is a neural network.

The second correspondence information is information in which material information, a process condition, and physical property information are associated with performance information. The second correspondence information is information acquired in advance by the information processing system 100 by a predetermined method such as a regression analysis. When the composition is a resist, the second correspondence information is, for example, information in which performance information indicating patterning performance of the resist, which is a resist processed by a process indicated by the process condition and is composed of a material indicated by the material information, is associated with physical property information thereof. Processing indicated by the process condition is, for example, processing using the resist. The processing indicated by the process condition may be, for example, processing of drying the resist according to a heating process after performing predetermined processing on the resist. The processing indicated by the process condition may also be, for example, processing of chemically changing the resist after executing predetermined processing on the resist. The predetermined processing for the resist is, for example, processing of applying the resist to an application target. The second correspondence information is, for example, a regression model that is acquired in a regression analysis method such as multiple regression, PCA regression, Lasso regression, Ridge regression, Elastic Net regression, partial least squares (PLS) regression, support vector regression, or the like, and is a regression model having material information, a process condition, and physical property information as explanatory variables and performance information as an objective variable.

A flow of processing until the information processing system 100 acquires performance information is as follows when it is described using the first correspondence information and the second correspondence information. That is, the information processing system 100 first acquires physical property information on the basis of the input material information and process condition and the first correspondence information. Next, the information processing system 100 acquires performance information on the basis of the input material information and process condition, the physical property information, and the second correspondence information. The first correspondence information is, for example, a learned model (hereinafter referred to as a “first model”) having material information and a process condition as explanatory variables and physical property information as an objective variable. The first correspondence information may be, for example, a relational database which shows a relationship between material information and a process condition and physical property information indicating the physical properties of a resist measured in a process with the material information and the process condition. The second correspondence information is, for example, a regression model (hereinafter referred to as a “second model”) having material information, a process condition, and physical property information as explanatory variables and performance information as an objective variable. The second correspondence information may be, for example, a relational database which shows a relationship between material information, a process condition, and physical property information and performance information.

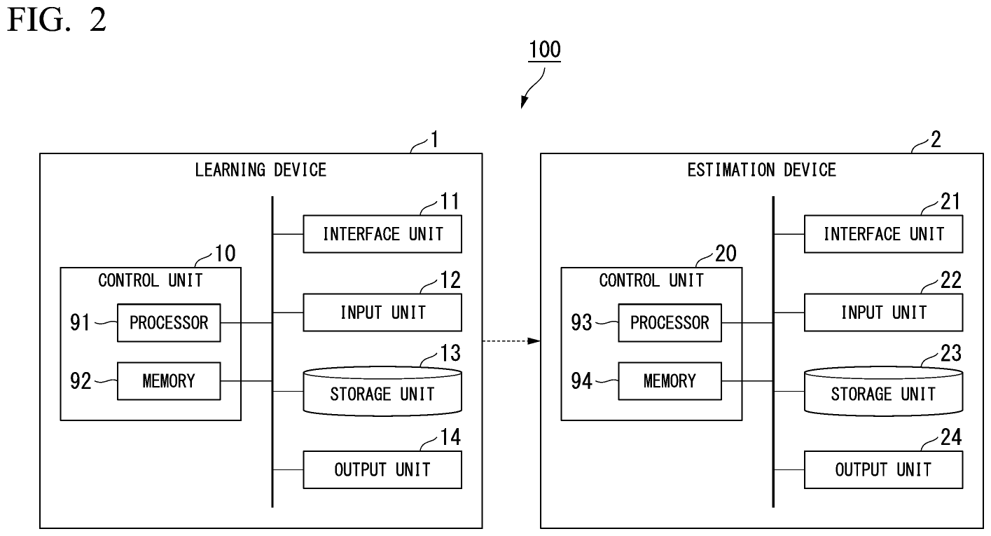

FIG. 2 is a diagram which shows an example of a system configuration of the information processing system 100 according to the embodiment. In the following description, for ease of understanding, the information processing system 100 will be described by taking a case where the composition is a resist and the performance is patterning performance as an example. In addition, the information processing system 100 will be described below by taking a case where the first correspondence information is the first model as an example. Moreover, the information processing system 100 will be described below by taking a case where the second correspondence information is the second model as an example.

The information processing system 100 includes a learning device 1 and an estimation device 2. The learning device 1 learns the first correspondence information. The learning device 1 includes a control unit 10 including a processor 91 such as a central processing unit (CPU) and a memory 92 connected by a bus, and executes a program. The learning device 1 functions as a device including a control unit 10, an interface unit 11, an input unit 12, a storage unit 13, and an output unit 14 by executing the program. More specifically, the processor 91 reads a program stored in the storage unit 13, and stores the read program in the memory 92. When the processor 91 executes a program stored in the memory 92, the learning device 1 functions as a device including the control unit 10, the interface unit 11, the input unit 12, the storage unit 13, and the output unit 14.

The interface unit 11 is configured to include a communication interface for connecting a host device to the estimation device 2 and an external device. The interface unit 11 communicates with the estimation device 2 and the external device in a wired or wireless manner.

The input unit 12 is configured to include an input device such as a mouse, a keyboard, or a touch panel. The input unit 12 may be configured as an interface for connecting these input devices to the host device. The input unit 12 receives an input of various types of information on the host device. The input unit 12 receives, for example, an input of learning data.

The storage unit 13 is configured by using a non-temporary computer readable storage medium device such as a magnetic hard disk device or a semiconductor storage device. The storage unit 13 stores various types of information related to the learning device 1. The storage unit 13 stores learning data input via the input unit 12. The storage unit 13 stores, for example, a machine learning model before the end condition is satisfied. The storage unit 13 stores information (hereinafter referred to as “regression data”) in which learning data is associated with performance information.

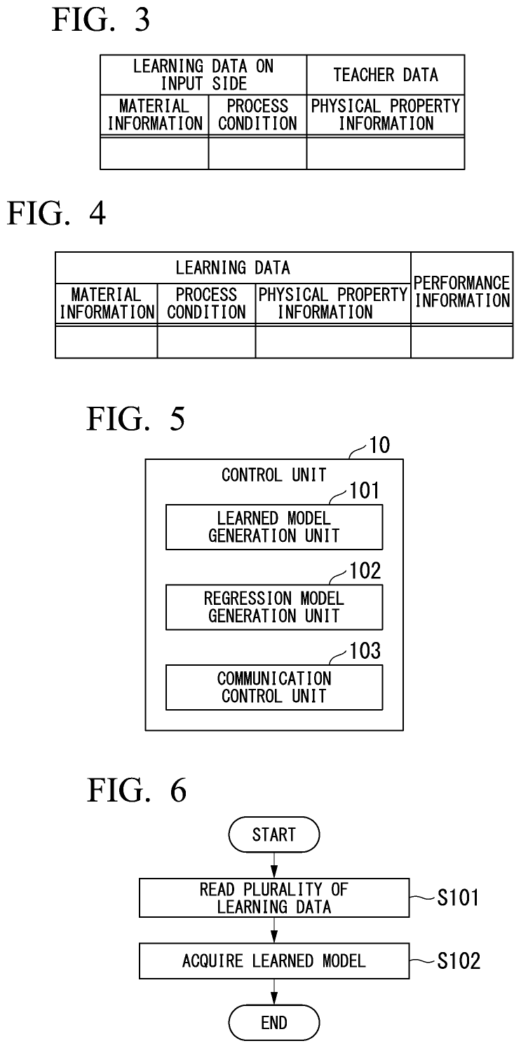

FIG. 3 is a diagram which shows an example of learning data in the embodiment.

The learning data is obtained by associating respective items of learning data on an input side and teacher data. The learning data on an input side stores respective types of information such as material information and a process condition. The teacher data stores physical property information.

FIG. 4 is a diagram which shows an example of regression data in the embodiment.

The regression data is obtained by associating, for example, learning data with performance information. The learning data stores respective types of information such as material information, a process condition, and physical property information.

The output unit 14 outputs various types of information. The output unit 14 outputs, for example, the first correspondence information of a learning result. The output unit 14 is configured to include, for example, a display device such as a cathode ray tube (CRT) display, a liquid crystal display, or an organic electro-luminescence (EL) display. The output unit 14 may be configured as an interface for connecting these display devices to the host device.

The control unit 10 controls an operation of each functional unit included in the learning device 1. In addition, the control unit 10 generates a first model and a second model.

FIG. 5 is a diagram which shows an example of a functional configuration of the control unit 10 in the embodiment. The control unit 10 includes a learned model generation unit 101, a regression model generation unit 102, and a communication control unit 103.

The learned model generation unit 101 reads a plurality of learning data stored in the storage unit 13. The learned model generation unit 101 generates a first model on the basis of a plurality of learning data. The generation of the first model means reading a machine learning model stored in the storage unit 13 and learning using a plurality of learning data until the end condition is satisfied. The learned model generation unit 101 stores the first model in the storage unit 13.

The regression model generation unit 102 reads a plurality of regression data and the first model stored in the storage unit 13. The regression model generation unit 102 generates a second model on the basis of the plurality of regression data and the first model. The generation of a second model means executing a predetermined regression analysis on the plurality of regression data and acquiring a regression model.

The communication control unit 103 controls an operation of the interface unit 11 and transmits the first model generated by the learned model generation unit 101 and the second model generated by the regression model generation unit 102 to the estimation device 2.

FIG. 6 is a flowchart which shows an example of a flow of processing of generating a first model executed by the learning device 1 in the embodiment.

The learned model generation unit 101 reads a plurality of learning data from the storage unit 13 (step S101). The learned model generation unit 101 performs machine learning on the basis of the plurality of read learning data, and generates a first model (step S102).

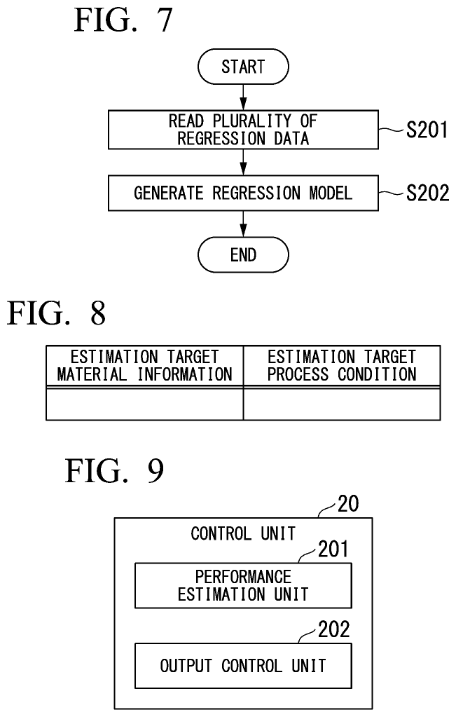

FIG. 7 is a flowchart which shows an example of a flow of processing of generating a second model executed by the learning device 1 in the embodiment.

The regression model generation unit 102 reads a plurality of regression data from the storage unit 13 (step S201). The regression model generation unit 102 executes a predetermined regression analysis on the plurality of read regression data and generates a second model (step S202).

Returning to the description in FIG. 2, the estimation device 2 includes a control unit 20 including a processor 93 such as a central processing unit (CPU) and a memory 94 connected by a bus, and executes a program. The estimation device 2 functions as a device including a control unit 20, an interface unit 21, an input unit 22, a storage unit 23, and an output unit 24 by executing the program. More specifically, the processor 93 reads a program stored in the storage unit 23, and stores the read program in the memory 94. When the processor 93 executes the program stored in the memory 94, the estimation device 2 functions as the device including the control unit 20, the interface unit 21, the input unit 22, the storage unit 23, and the output unit 24.

The interface unit 21 is configured to include a communication interface for connecting the host device to the learning device 1 and an external device. The interface unit 21 communicates with the learning device 1 and the external device in a wired or wireless manner.

The input unit 22 is configured to include an input device such as a mouse, a keyboard, or a touch panel. The input unit 22 may be configured as an interface for connecting these input devices to a host device. The input unit 22 receives an input of various types of information on the host device. The input unit 22 receives, for example, an input of estimation target information. The estimation target information is the information in which estimation target material information is associated with an estimation target process condition. The estimation target material information is material information indicating a material of a composition for which the estimation device 2 estimates patterning performance. The estimation target process condition is a process condition of a process for obtaining the composition for which the estimation device 2 estimates patterning performance.

The storage unit 23 is configured by using a non-temporary computer readable storage medium device such as a magnetic hard disk device or a semiconductor storage device. The storage unit 23 stores various types of information on the estimation device 2. The storage unit 23 stores, for example, correspondence information. That is, the storage unit 23 stores, for example, the first model and the second model. The storage unit 23 stores, for example, the estimation target information input via the input unit 22.

FIG. 8 is a diagram which shows an example of the estimation target information in the embodiment.

The estimation target information is obtained by associating respective items of the estimation target material information and the estimation target process conditions.

The output unit 24 outputs various types of information. The output unit 24 outputs, for example, performance information that is a result of estimation by the estimation device 2. The output unit 24 is configured to include, for example, a display device such as a cathode ray tube (CRT) display, a liquid crystal display, and an organic electro-luminescence (EL) display. The output unit 24 may be configured as an interface for connecting these display devices to a host device.

The control unit 20 estimates patterning performance obtained by a process under the estimation target process condition of a resist composed of a material indicated by the estimation target material information on the basis of correspondence information.

FIG. 9 is a diagram which shows an example of a functional configuration of the control unit 20 in the embodiment. The control unit 20 includes a performance estimation unit 201 and an output control unit 202.

The performance estimation unit 201 reads estimation target information and correspondence information stored in the storage unit 23. The performance estimation unit 201 acquires performance information indicating performance of a composition indicated by the estimation target information, which is performance obtained by a process under a process condition indicated by the estimation target information, on the basis of the correspondence information.

The output control unit 202 controls an operation of the output unit 24 and outputs the performance information acquired by the performance estimation unit 201 to the output unit 24.

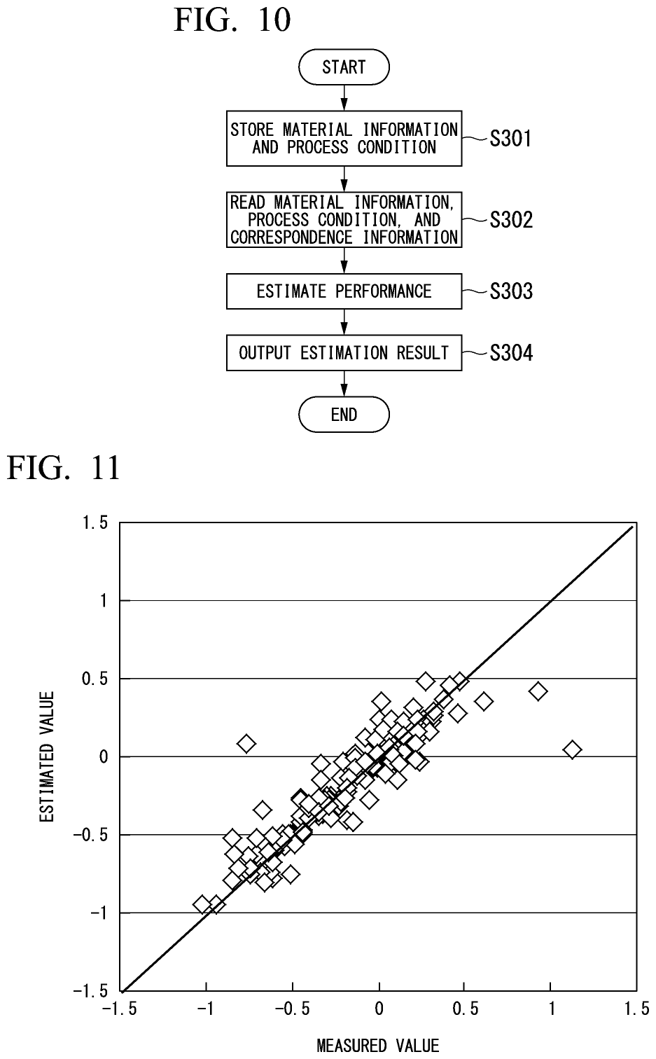

FIG. 10 is a flowchart which shows an example of a flow of processing executed by the estimation device 2 in the embodiment.

The storage unit 23 stores estimation target information input via the input unit 22 (step S301). Next, the performance estimation unit 201 reads the estimation target information and the correspondence information stored in the storage unit 23 (step S302). Next, the performance estimation unit 201 estimates the performance of a composition indicated by the estimation target information, which is the performance obtained by a process under a process condition indicated by the estimation target information, on the basis of the correspondence information (step S303). For example, the performance estimation unit 201 first acquires physical property information corresponding to the estimation target information on the basis of the estimation target information and first correspondence information. Next, the performance estimation unit 201 acquires performance information corresponding to estimation target information and physical property information corresponding to the estimation target information on the basis of the estimation target information, the physical property information corresponding to the estimation target information, and second correspondence information. The performance information acquired in this manner is a result of the estimation that is processing of step S303. Next to step S303, the output control unit 202 outputs performance that is the result of the estimation to the output unit 24 (step S304).

FIG. 11 is a diagram which shows a first example of an experimental result that shows a relationship between a result of estimating performance information by the information processing system 100 in the embodiment and an actually measured performance.

A horizontal axis of FIG. 11 shows a measured value, and a vertical axis shows a value (an estimated value) of the result of the estimation. FIG. 11 shows that root mean squared error (RMSE) is 0.1747. FIG. 11 shows that a correlation coefficient is 0.8863. FIG. 11 shows that a coefficient of determination is 0.7855. The RMSE of 0.1747, the correlation coefficient of 0.8863 and the coefficient of determination of 0.7855 indicate that the information processing system 100 can estimate performance with reliable and high accuracy in development of a new composition.

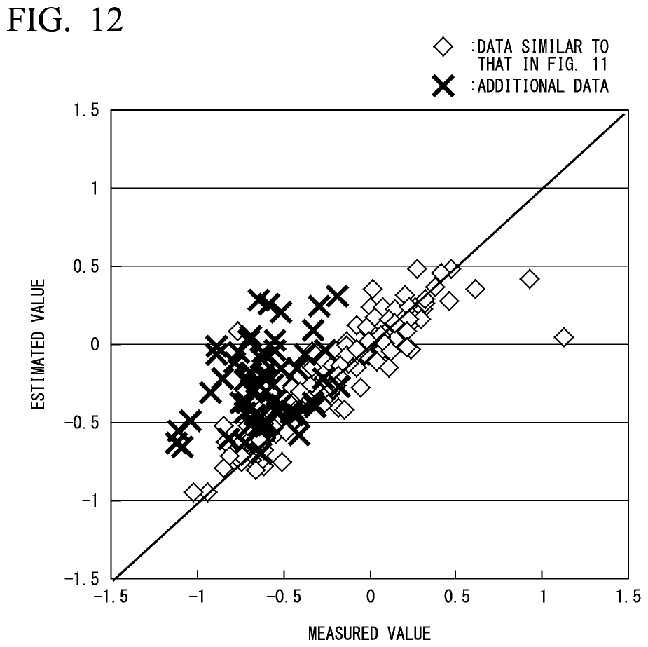

FIG. 12 is a diagram which shows a second example of the experimental result that shows the relationship between a result of estimating performance information by the information processing system 100 in the embodiment and an actually measured performance.

A horizontal axis of FIG. 12 shows a measured value, and a vertical axis shows a value (an estimated value) of the result of the estimation. FIG. 12 shows a result when further data is added in addition to the data of FIG. 11. FIG. 12 also shows that the information processing system 100 can estimate performance with reliable and high accuracy in the development of a new composition.

The information processing system 100 of the embodiment configured in this manner estimates performance obtained by a process under the process condition of a composition composed of a material indicated by target material information on the basis of correspondence information. For this reason, the labor of developers developing new compositions can be reduced.

### Modified Example

The material information may include, for example, a surface area of a molecule serving as a material, a volume of the molecule serving as a material, a molecular weight of the molecule serving as a material, a value representing a charge density distribution of the molecule serving as a material, a value representing a molecular descriptor, a molar thermal capacity of a material, a coefficient of thermal expansion of the material, a dielectric constant of the material, a surface tension of a material, a viscosity of a material, a refractive index of a material, a transmittance of a material, an absorbance of a material, a density of a material, a glass transition temperature of a material, a melting point of a material, a distribution coefficient of a material, an acidity constant of a material, a solubility parameter of a material, ABC parameters of a material described in Reference 1 below, or an activation energy of a deprotection reaction of a protective group of a material.
Reference 1: F. H. Dill, A. R. Neureuther, J. A. Tuttle and E. J. Walker “Modeling projection printing of positive photoresists,” IEEE Trans. Electron. Dev., 22, (1975), pp. 456-464.

The physical property information may be any type of information as long as it is used for outputting performance information on the basis of the second correspondence information. The physical property information may be, for example, information indicating physical properties of a resist which is a resist before and/or after being processed in a process under a process condition, and is a material indicated by the material information. More specifically, the physical property information may be, for example, characteristic information of a protective film formed on a predetermined target as a result of processing the resist. The processing for the resist is, for example, processing of applying the resist to an application target. In such a case, the predetermined target on which the protective film is formed is an application target to which the resist is applied. More specifically, the physical property information may be, for example, the characteristic information of the protective film formed on the predetermined target as a result of processing the resist and drying the resist according to the heating process. The physical property information may be, for example, the characteristic information of a protective film formed on a predetermined target as a result of processing the resist and chemically changing the resist.

The physical property information may include at least one type of information selected from a group made of, for example, the surface area of a molecule serving as a material, the volume of the molecule serving as a material, the molecular weight of the molecule serving as a material, the value representing the charge density distribution of the molecule serving as a material, the value representing a molecular descriptor, the molar thermal capacity of a material, the coefficient of thermal expansion of a material, the dielectric constant of a material, the surface tension of a material, the viscosity of a material, the refractive index of a material, the transmittance of a material, the absorbance of a material, the density of a material, the glass transition temperature of a material, the melting point of a material, a boiling point of a material, a flash point of a material, a vapor pressure of a distribution coefficient of a material, onishi parameters of a material, a pKa value of a material, a decomposition point of a material, a distribution coefficient of a material, the acidity constant of a material, the solubility parameter of a material, the ABC parameters of a material described in Reference 1 below, the activation energy of the deprotection reaction of the protective group of a material, an acid diffusion length of a material, the molecular weight of a polymer serving as a material, a molecular weight dispersion of the polymer serving as a material, information indicating a composition ratio of a polymer material (a polymer unit) serving as a material, information indicating the amount of added components such as a photoacid generator (PAG) and a photodisintegrating base (PDB), information indicating a dissolution rate of a resist film in an unexposed state when the composition is a resist, information indicating the dissolution rate of the resist film in an exposed state when the composition is a resist, and information obtained by comparing between states of the resist film before and after being exposed when the composition is a resist. The information obtained by comparing between the states of the resist film before and after being exposed when the composition is a resist is, for example, a change in film thickness, weight, film density, dissolution rate, refractive index, and the like.

The process condition may include, for example, at least one type of information selected from a group made of an applied film thickness, a thermal treatment condition, an exposure condition, an observation condition using an electron microscope, mask information, and a normalized image log-slope (NILS). The mask information is information on a photomask. The thermal treatment condition may include, for example, a temperature of post applied bake (PAB), a temperature of post exposure bake (PEB), and temperature and time conditions for a bake such as PAB and PEB. Specifically, the observation condition by the electron microscope is an observation magnification, a current value, an acceleration voltage, a number of frames, or the like.

The patterning performance is, for example, at least one type of information selected from a group made of sensitivity, critical dimension uniformity (CDU), limit resolution, line edge roughness (LER), line width roughness (LWR), depth of focus (DOF), exposure margin (EL margin), mask error factor (MEF), rectangularity of a pattern cross-sectional shape, and roundness of a hole in a contact hole pattern (a CH pattern).

The first correspondence information does not necessarily have to be the first model, but is desirable to be a non-linear model that shows a relationship between material information and a process condition and physical property information indicating the physical properties of a resist measured in a process under the material information and the process condition.

The second correspondence information does not necessarily have to be the second model, but is desirable to be information acquired in a method with higher extrapolation accuracy than a method of generating the first correspondence information. For example, the second correspondence information is preferably a linear model that shows a relationship between material information, a process condition, and physical property information and performance information indicating patterning performance. The linear model may be, for example, multiple regression, PCA regression, Lasso regression, Ridge regression, Elastic Net regression, partial least squares (PLS) regression, or support vector regression.

In the embodiment, the learned model generation unit 101 has generated the first model, but the first model is merely an example of the first correspondence information, and the learned model generation unit 101 is a functional unit that generates the first correspondence information. In addition, the regression model generation unit 102 has generated the second model in the embodiment, but the second model is merely an example of the second correspondence information, and the regression model generation unit 102 is a functional unit that generates the second correlation information. Moreover, the performance estimation unit 201 has estimated the performance information on the basis of the first model and the second model in the embodiment. However, the first model and the second model are merely examples of the first correspondence information and the second correspondence information, and the performance estimation unit 201 is a functional unit that estimates the performance information on the basis of the first correspondence information and the second correspondence information.

The learning data may be input by an external device via the interface unit 11. The estimation target information may be input by an external device via the interface unit 21.

All or a part of functions of the learning device 1 and the estimation device 2 may be realized by using hardware such as an application specific integrated circuit (ASIC), a programmable logic device (PLD), and a field programmable gate array (FPGA). A program may be recorded on a computer-readable recording medium. The computer readable recording medium is, for example, a flexible disk, a magneto-optical disc, a portable medium such as a ROM or a CD-ROM, or a storage device such as a hard disk embedded in the computer system. The program may be transmitted via a telecommunication line.

Note that the learning device 1 and the estimation device 2 may be implemented by using a plurality of information processing devices connected to be able to communicate with each other via a network. In this case, each functional unit included in the learning device 1 and the estimation device 2 may be distributed and implemented in a plurality of information processing devices. For example, the learned model generation unit 101 and the regression model generation unit 102 may be implemented in different information processing devices.

The learning device 1 and the estimation device 2 do not necessarily have to be mounted in different housings. The learning device 1 and the estimation device 2 may be devices composed of one housing. The estimation device 2 does not necessarily have to read correspondence information from the storage unit 23, but may read it from the storage unit 13 via the interface unit 11 and the interface unit 21.

The learned model generation unit 101 is an example of the learning unit.

As described above, the embodiment of the present invention has been described in detail with reference to the drawings, but the specific configuration is not limited to this embodiment, and the design and the like within a range not departing from the gist of the present invention are also included. Therefore, the scope of the present invention is defined only by the scope of claims and the equivalent scope thereof.

### REFERENCE SIGNS LIST

100 Information processing system
1 Learning device
2 Estimation device
10 Control unit
11 Interface unit
12 Input unit
13 Storage unit
14 Output unit
20 Control unit
21 Interface unit
22 Input unit
23 Storage unit
24 Output unit
91 Processor
92 Memory
93 Processor
94 Memory
101 Learned model generation unit
102 Regression model generation unit
103 Communication control unit

What is claimed is:

1. An information processing system comprising:
a processor; and
a storage medium configured to store correspondence information in which material information indicating a material of a composition and a process condition in a process using the composition are associated with performance information of a composition obtained by the process the storage medium having computer program instructions stored thereon, wherein the computer program instructions, when executed by the processor, perform processing of:
acquiring the performance information on the basis of the input material information and processing condition, and the correspondence information; and
outputting the performance information.

2. The information processing system according to claim 1,
wherein the composition is used for patterning of an object, and the performance information indicates a performance when the composition is used for the patterning.

3. The information processing system according to claim 1,
wherein the composition is a resist.

4. The information processing system according to claim 3,
wherein the storage medium stores first correspondence information and second correspondence information as the correspondence information,
the first correspondence information is information in which the material information and the process conditions are associated with physical property information indicating physical properties of the resist in a process under the process conditions,
the second correspondence information is information in which the material information, the process conditions, the physical property information, and the performance information are associated with each other, and
the computer program instructions perform processing of:
outputting the physical property information on the basis of the input material information, process condition, and the first correspondence information; and
acquiring the performance information on the basis of the output physical property information, material information, process conditions, and second correspondence information.

5. The information processing system according to claim 4,
wherein the first correspondence information is a non-linear model having the material information and the process condition set as explanatory variables and having the physical property information set as an objective variable.

6. The information processing system according to claim 4,
wherein the second correspondence information is represented by a linear model having the material information, the process conditions, and the physical property information as explanatory variables and having the performance information as an objective variable.

7. The information processing system according to claim 4, wherein the computer program instructions further perform processing of:
generating the first correspondence information by performing machine learning on the basis of the material information, a process condition, and physical property information indicating the physical properties of the resist in a process with the material information and the process condition.

8. The information processing system according to claim 7,
wherein the second correspondence information is information acquired in a method having a higher extrapolation accuracy than a method generating the first correspondence information by the machine learning.

9. The information processing system according to claim 4,
wherein the physical property information is information indicating a physical property of a resist that is a resist before and/or after being processed under the process conditions, and is a material indicated by the material information.

10. The information processing system according to claim 3, wherein the performance information is information indicating lithography performance of the resist.

11. The information processing system according to claim 9, wherein the physical property information is characteristic information of a protective film formed on a predetermined target as a result of processing the resist.

12. The information processing system according to claim 11, wherein the physical property information is characteristic information of a protective film formed on a predetermined target as a result of processing the resist and drying the resist by a heating process.

13. The information processing system according to claim 11, wherein the physical property information is characteristic information of a protective film formed on a predetermined target as a result of processing the resist and chemically changing the resist.

14. An information processing device comprising:
a processor; and
a storage medium having computer program instructions stored thereon, wherein the computer program instructions, when executed by the processor, perform processing of:
reading correspondence information in which material information indicating a material of a resist and a process condition in a predetermined process using the resist are associated with performance information indicating performance of the resist obtained by the process from a storage medium,
and acquiring the performance information on the basis of the read material information and process condition and the correspondence information; and
outputting the performance information.

15. A learning device comprising:
a processor; and
a storage medium having computer program instructions stored thereon, wherein the computer program instructions, when executed by the processor, perform processing of:
generating first correspondence information by performing machine learning on the basis of material information indicating a material of a resist and a process condition in a predetermined process using the resist, and physical property information indicating a physical property of the resist in a process with the material information and the process condition.

16. The learning device according to claim 15, wherein the physical property information associated with the material information and process condition in the first correspondence information is used to output performance information on the basis of second correspondence information in which the physical property information and the performance information indicating performance of the resist obtained by the process are associated with each other.

17. An information processing method comprising:
acquiring performance information on the basis of correspondence information in which material information indicating a material of a resist and a process condition in a predetermined process using the resist are associated with the performance information indicating performance of the resist obtained by the process; and
outputting the performance information.

18. (canceled)

19. A learning method comprising:
generating first correspondence information by performing machine learning on the basis of material information indicating a material of a resist and a process condition in a predetermined process using the resist, and physical property information indicating a physical property of the resist in a process with the material information and the process condition.

20. (canceled)
